(19) United States
(12) Patent Application Publication
(10) Pub. No.: US 2019/0187061 A1
(43) Pub. Date: Jun. 20, 2019

(54) METHODS AND APPARATUS FOR INFRARED AND MID-INFRARED SENSING

(71) Applicant: Massachusetts Institute of Technology, Cambridge, MA (US)

(72) Inventors: Zhaohong HAN, Cambridge, MA (US); Jianwei MU, Pleasanton, CA (US); Anuradha Murthy AGARWAL, Weston, MA (US); Pao Tai LIN, Brighton, MA (US); Lionel Cooper KIMERLING, Concord, MA (US)

(21) Appl. No.: 16/213,376

(22) Filed: Dec. 7, 2018

Related U.S. Application Data
(63) Continuation of application No. 15/425,443, filed on Feb. 6, 2017, now abandoned.
Application No. 15/425,443 is a continuation of application No. PCT/US2016/014892, filed on Jan. 26, 2016.
(60) Provisional application No. 62/107,549, filed on Jan. 26, 2015.

(51) Int. Cl. G01N 21/77; G01N 21/64; G01N 21/65; G01N 21/41; G01N 33/543

(52) U.S. Cl. CPC G01N 21/7746; G01N 21/648; G01N 21/65; G01N 21/41; G01N 33/54373; G01N 2021/7783; G01N 2021/7776; G01N 2021/7786; G01N 2021/7789; G01N 2201/061; G01N 2021/655

(57) ABSTRACT

A sensing apparatus includes a light source to transmit a light beam, an input switch, a first sensing element, a second sensing element, and a detector. The input switch receives the light beam and includes a phase change material having a first state and a second state. The first sensing element receives the light beam from the input switch when the phase change material is in the first state and produces a first change in the light beam in response to a presence of a first analyte. The second sensing element receives the light beam from the input switch when the phase change material is in the second state and produces a second change in the light beam in response to a presence of a second analyte. The detector detects the first change and/or the second change in the light beam.

### CROSS-REFERENCE TO RELATED APPLICATIONS

This application is a continuation of U.S. application Ser. No. 15/425,443, filed Feb. 6, 2017, entitled “Methods and Apparatus for Infrared and Mid-Infrared Sensing,” which is a continuation of International Patent Application No. PCT/US2016/014892, filed Jan. 26, 2016, entitled “Methods and Apparatus for Infrared and Mid-Infrared Sensing,” which in turn claims priority to U.S. Application No. 62/107,549, filed Jan. 26, 2015, entitled “Monolithic Energy-Efficient Scalable Mid-IR Sensor.” Each of these applications is hereby incorporated herein by reference in its entirety.

### BACKGROUND

Infrared (IR) and mid-IR sensing (e.g., at wavelengths of about 1 μm to about 40 μm) is a domain of interest to advanced material metrology in many areas because a large number of molecules can undergo strong characteristic vibrational transitions in this wavelength region. On-chip IR and mid-IR sensors can be helpful for fast and accurate detection of these molecules. It can also be helpful for one sensor chip to have multiple functions, such as sensing more than one type of molecules, or sensing more than one characteristic vibrational transition in the same molecule to more accurately detect the molecule.

Existing methods to achieve multiple functions in IR and mid-IR sensor chips fall into two general categories. In one category, multiple light sources are used. Each light source is coupled to a sensing waveguide or resonator that is sensitive to a particular wavelength of light, and a detector is then coupled to the sensing waveguide or resonator to detect the light. However, light sources and detectors are usually expensive, therefore increasing the costs of these on-chip sensors. In addition, on-chip sensors having multiple light sources and detectors can be energy consuming since each light source and detector normally have their own power source.

In the second category, a broadband light source is employed to deliver a broadband light beam, which is then split into multiple channels. Each channel includes a sensing waveguide or resonator that is sensitive to a particular wavelength. This category of device may use only one light source and one detector. However, the light intensity at each channel is normally low because of the beam splitting. Low intensity can limit the sensitivity of the sensor. In addition, light beams split into different sensing channels are usually combined again at the detector. One beam from one sensing channel may therefore interfere with another beam from a different sensing channel, creating challenges in analyzing the detected signals.

### SUMMARY

Embodiments of the present invention include apparatus, systems, and methods of infrared and mid-infrared sensing. In one example, an apparatus for sensing a first analyte and a second analyte includes a light source to transmit a light beam, an input switch, a first sensing element, a second sensing element, and a detector. The input switch is in optical communication with the light source to receive the light beam from the light source. The input switch includes a phase change material having a first state and a second state. The first sensing element is in optical communication with the input switch to receive the light beam from the input switch when the phase change material is in the first state. The first sensing element produces a first change in the light beam in response to a presence of the first analyte. The second sensing element is also in optical communication with the input switch and receives the light beam from the input switch when the phase change material is in the second state. The second sensing element produces a second change in the light beam in response to a presence of the second analyte. The detector is in optical communication with the first sensing element and the second sensing element to detect at least one of the first change in the light beam and the second change in the light beam.

In another example, a method of sensing a first analyte and/or a second analyte includes transmitting a light beam to an input switch that includes a phase change material. The light beam is then transmitted from the input switch to a first sensing element when the phase change material is in a first state. A presence of the first analyte is detected based on a first change in the light beam transmitted via the first sensing element. The method also includes changing the phase change material from the first state to a second state and transmitting the light beam to a second sensing element when the phase change material is in the second state. A presence of the second analyte is detected based on a second change in the light beam transmitted via the second sensing element.

In yet another example, an apparatus for mid-infrared sensing includes a substrate, on which a broadband light source is disposed to emit a light beam having at least one spectral component at a wavelength in a range of about 2 μm to about 7 μm. An input switch is also disposed on the substrate in optical communication with the broadband light source to receive the light beam. The input switch includes a phase change material switchable between a transmissive state and an absorptive state. An actuator is operably coupled to the input switch to switch the phase change material between the transmissive state and the absorptive state. The apparatus also includes a first sensing element disposed on the substrate in optical communication with the input switch to receive the light beam from the input switch when the phase change material is in the transmissive state. The apparatus further includes a second sensing element, disposed on the substrate in optical communication with the input switch, to receive the light beam from the input switch when the phase change material is in the absorptive state. A detector is disposed on the substrate in optical communication with the first sensing element and the second sensing element to receive the light beam from the first sensing element when the phase change material is in the transmissive state and to receive the light beam from the second sensing element when the phase change material is in the absorptive state.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 shows a schematic of a sensing apparatus including a switch using a phase change material.

FIG. 2 shows a perspective view of a network of sensors coupled by switches using phase change materials.

FIG. 3 shows a perspective view of a switch using a phase change material.

FIGS. 4A-4B show schematics of switches including a phase change material and an actuator to control the phase of the phase change material.

FIG. 5A shows a perspective view of a SiNₓ waveguide.

FIG. 5B shows a cross sectional view of the SiNₓ waveguide shown in FIG. 5A.

FIGS. 6A-6B are simulated results of beam profiles of light beams at 2.45 μm and 2.75 μm, respectively, in the SiNₓ waveguides shown in FIGS. 5A-5B.

FIGS. 7A-7B line profiles along the diameter of the light beams shown in FIGS. 6A-6B.

FIG. 8 shows a perspective view of a directional coupler including SiNₓ waveguides shown in FIGS. 5A-5B.

FIG. 9 shows calculated output intensity distributions from two channels in the directional coupler shown in FIG. 8.

FIGS. 10A-10C are simulated 2D intensity profiles at three different wavelengths 2.42 μm, 2.55 μm, and 2.65 μm, respectively.

FIGS. 11A-11C show Scanning Electron Microscope (SEM) images of silicon nitride waveguides and directional couplers.

FIG. 12 shows a schematic of a switch including Germanium Antimony Telluride (GST) sandwiched by two gold electrodes to control the phase of the GST.

FIGS. 13A-13B are simulated results of optical field distribution in the switch shown in FIG. 12.

FIGS. 14A-14B show simulated insertion losses of the switch shown in FIG. 12 as a function of thickness and waveguide width, respectively.

FIG. 15A shows the power density to control the switch shown in FIG. 12.

FIG. 15B shows the total energy to control the switch shown in FIG. 12.

### DETAILED DESCRIPTION

Sensing Apparatus including a Switch using Phase Change Materials

Apparatus and methods described herein address, at least partially, the challenges in conventional approaches to develop multifunctional on-chip IR and mid-IR sensors. Example apparatus employ a switch having a phase change material to couple two sensing elements to a light source. Light beams delivered by the light source can be transmitted to one of the sensing elements or the other by adjusting the state of the phase change material (e.g., via heating the phase change material). For example, the phase change material can be Germanium Antimony Telluride (GST) which has an amorphous state and a crystalline state. When the GST is in the amorphous state, light beams can be transmitted to one sensing element. When the GST is in the crystalline state, light beams can then be transmitted into the other sensing element. In either case, nearly all the beam energy (e.g., >95%) from the light source is delivered into one sensing element for sensing. Therefore, the sensitivity in this apparatus can be high. In addition, during detection, the detector can receive the light beam from one sensing element, thereby reducing potential interference between light from different channels. Since a single light source and detector are used, the total cost and energy consumption can also be decreased and the compactness and portability of the sensor can be improved. Furthermore, phase change materials can usually stay at the same state after adjustment without additional energy input. Therefore, operation of the switches may only consume energy during switching from one state to another, so the total energy consumption can still be low.

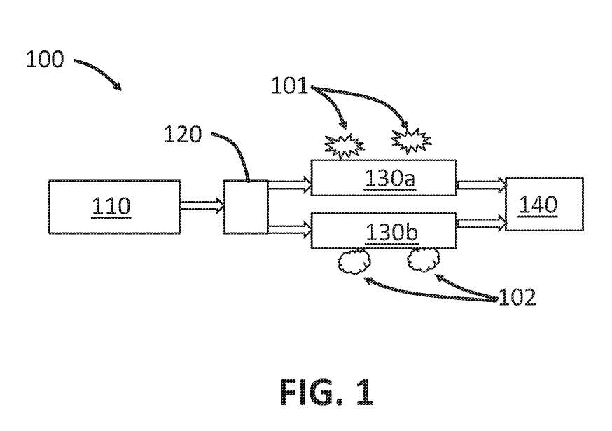

FIG. 1 shows a schematic of a sensing apparatus 100 that can detect more than one type of analyte or more than one characteristic transition using a switch. The apparatus 100 includes a light source 110 to transmit a light beam. An input switch 120 is in optical communication with the light source 110 to receive the light beam from the light source 110. The input switch 120 includes a phase change material having two states generally referred to as a first state and a second state. Two sensing elements 130a and 130b (collectively referred to as sensing elements 130) are optically coupled to the input switch 120 to receive the light beam. When the phase change material in the input switch 120 is in the first state, the light beam is transmitted to the first sensing element 130a. The first sensing element can detect the presence (or the absence) of an analyte 101 by producing a change in the light beam in response to the presence of the analyte 101. On the other hand, when the phase change material in the input switch 120 is in the second state, the light beam is transmitted to the second sensing element 130b, which can detect another analyte 102 by producing a change in the light beam in response to the presence of the analyte 102. The light beam transmitted through either the first sensing element 130a or the second sensing element 130b is then detected by a detector 140, which can sense the change in the light beam and therefore determines whether the analyte 101 or 102 is present.

The light source 110 may include one or more narrowband/coherent emitters (e.g., quantum cascade lasers), broadband emitters, or tunable emitters to emit a beam of mid-IR light. For example, the light source may emit a beam at a wavelength that can span or be tuned over some or all of the mid-IR portion of the electromagnetic spectrum, e.g., over a bandwidth of about 1.0 μm to about 40 μm (e.g., about 2 μm, 5 μm, 10 μm, 15 μm, 20 μm, 25 μm, 30 μm, 35 μm, or 40 μm).

The wavelength of the light emitted by the light source 110 can be tuned or chosen based on known characteristic absorption bands to monitor a fluid for one or more particular chemicals. For example, hexane has a distinguishable absorbance at λ=3.55 μm, so tuning the probe light to this wavelength yields a signal that can be used to detect hexane relatively easily. Likewise, to detect a compound that includes an amine functional group, the probe beam's wavelength may be between λ=2.85 μm and λ=3.22 μm to interrogate absorption from the N—H stretch associated with the amine functional group.

The input switch 120 includes the phase change material that can be switched between the first state and the second state. In general, the phase change material in one of the states is transmissive to the light beams and becomes absorptive when switched to the other state. For example, Germanium Antimony Telluride (GST) can have an amorphous state (transmissive state) and a crystalline state (absorptive state). The refractive index of GST changes significantly during the phase change process. Without being bound by any particular theory of mode of operation, the phase change can be considered to arise from the significant variation in bonding between the crystalline phases and the amorphous phases. In the crystalline phases, the optical dielectric constant is strongly enhanced by resonant bonding effects, while that of the amorphous phases is more like a covalent semiconductor.

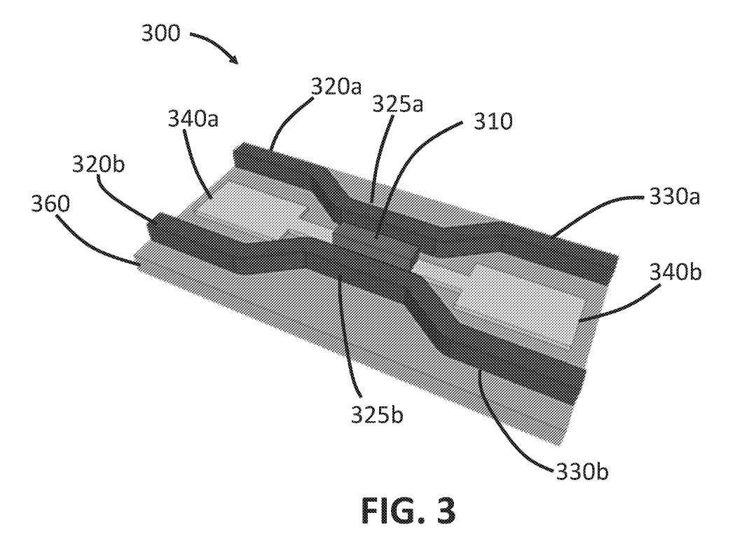

The input switch 120 can have at least two configurations (e.g., as shown in FIG. 3). In one example, the input switch 120 can have a directional coupler configuration. As understood in the art, directional couplers normally include two substantially parallel waveguides, each of which is coupled to an output channel. Light beams propagating in one waveguide can be coupled to the other waveguide (and therefore the output channel coupled to the waveguide) depending on the optical properties of the two waveguides and the material separating the waveguides. In the input switch 120, phase change materials can be disposed between the two waveguides. When the phase change material is in the transmissive state, light beams are allowed to be coupled from one waveguide to the other (i.e., the switch is ON). However, when the phase change material is in the absorptive state, light beams propagating in one waveguide may not be coupled into the other waveguide (i.e., the switch is OFF).

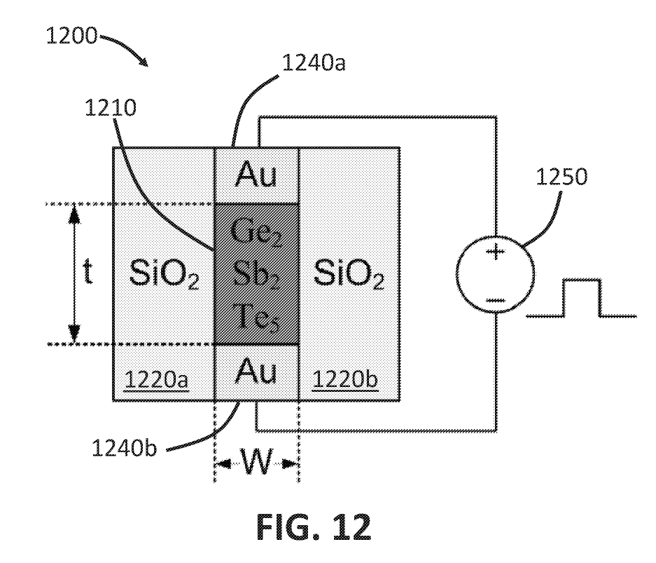

In another example, the input switch 120 can have a latch switch configuration (e.g., as shown in FIG. 12). A latch switch can be used to open or close certain channel. Phase change materials can be fabricated as part of the waveguide to allow or prevent the propagation of light beams. Switching the state of the phase change material can change the absorption of the waveguide system, thereby turning the channel on or off.

Various phase change materials can be used to construct the input switch 120. In one example, the input switch includes GST, which can have good phase-change repeatability and large optical differences between the amorphous phase and crystalline phase. In addition, GST can have low absorption in mid-IR range. GST is also stable at room temperature in both phases, which means no extra energy is needed to keep it in a given status.

In another example, the input switch 120 includes a Mott insulator (e.g., VO₂) that can be switched between a metal phase and an insulator phase. In yet another example, the input switch 120 can include a phase change material including one or more of AgInSbTe, InSe, SbSe, AsSe, GeSbSe, InSbTe, AgInSbSeTe, and GeSbTeSe. In yet another example, the input switch 120 can include a combination of one or more of the above mentioned materials.

The input switch 120 can be turned on and off by switching the phase or state of the phase change material. In general, the phase can be switched via temperature change; various methods can be used to change the temperature of the input switch 120. In one example, the phase of the phase change material can be switched using micro-scale hot plate nearby the input switch 120. In another example, the phase change can be obtained by direct current injection through the phase change material.

In yet another example, a phase transition in the phase change materials can be triggered via mechanical actuation, optical actuation (e.g., plasmonic absorption using meta-material or photonic crystal), electric field driven (non-heating) transformation, resistive heating, laser annealing/heating, or magnetic actuation.

More details and additional examples of switches that can be used in the apparatus 100 are described below with reference to FIGS. 3-15.

The sensing elements 130 in general propagate the light beams and the presence of analytes can produce changes (e.g., in intensity, spectrum, or phase) in the light beams. In one example, the sensing elements 130 include linear waveguides (e.g., straight semiconductor waveguides) that substantially confine light beams. Using linear waveguides in the sensing elements 130 can be helpful when broadband light beams are used, because linear waveguides can generally propagate light beams within a broad range of wavelengths (e.g., 1 μm to about 20 μm).

In another example, the sensing elements 130 include cavity resonators, which can be built by disposing two reflectors (e.g., Bragg gratings) on both ends of a linear waveguide. Light beams can propagate multiple times within the cavity so as to increase the interaction between the light beams and possible analytes nearby the sensing elements 130. In yet another example, the sensing elements 130 include ring resonators, which also allow multiple passes of light beams and therefore have increased sensitivity.

The linear waveguides or resonator waveguides in the sensing elements 130 can be disposed on pedestals to increase sensitivity. In one example, the sensing elements 130 can include silicon or germanium waveguides disposed on pedestals to reduce optical losses in the spectral region above, for example, 3 μm. More information of these pedestal waveguides can be found in U.S. Pat. No. 9,046,650, which is incorporated herein in its entirety.

In yet another example, the ring resonators are disposed on a pedestal to increase the coupling between the analyte and the optical field within or near the sensing elements 130. More details of pedestal ring resonators can be found in later material Application No. PCT/US2015/062590, which is incorporated herein in its entirety.

Various materials can be used to construct the sensing elements 130. In one example, the sensing elements 130 include silicon, such as single crystalline silicon (also referred to as sc-Si), multicrystalline silicon (also referred to as mc-Si), polycrystalline silicon (also referred to as pc-Si), amorphous silicon, or any other silicon known in the art.

In another example, the sensing elements 130 include chalcogenide glasses, which are generally based on chalcogen elements, such as sulphur, selenium, tellurium, and polonium. These glasses can be formed by the addition of other elements, such as germanium, arsenic, antimony, or gallium. Various types of chalcogenide glasses can be used for the sensing elements 130. For example, the sensing elements 130 can include pure chalcogenide (e.g., S, Se, Te, SₓSe₁₋ₓ). In another example, the sensing elements 130 can include pnictogen-chalcogen, such as (V-VI) As₂S₃ or P₂Se. In yet another example, the sensing elements 130 can include tetragen-chalcogen, such as (IV-VI) SiSe₂, GeS₂ III-VI B2S3, or InₓSe₁₋ₓ. In yet another example, the sensing elements 130 can include metal chalcogenide such as MoS₃, WS₃, or Ag₂S—GeS₂. In yet another example, the sensing elements 130 can include halogen-chalcogenide, such as As—Se—I, Ge—S—Br, or Te—Cl.

In yet another example, the sensing elements 130 include germanium. In yet another example, the sensing elements can include silicon-based materials, such as silicon nitride SiNₓ (e.g., Si₃N₄ or other stoichiometric silicon nitrides), silicon oxide SiOₓ (e.g., SiO or SiO₂), or any other silicon-based materials known in the art.

Several types of sensing can be carried out by the sensing elements 130. In one example, the sensing elements 130 can be configured to perform refractive index (RI) sensing. When analytes are present in the evanescent field of light propagating through the sensing elements 130, the refractive index experienced by the propagation modes in the sensing elements 130 can change because analytes usually have RIs different from that of the surrounding medium. This RI change can lead to a spectral shift in the propagation modes, which can be detected directly or indirectly (through intensity or phase detection) as indication of the presence of analytes.

In another example, the sensing elements 130 can be configured to perform fluorescence sensing. In general, photons in the sensing elements 130 can excite the analytes so as to induce fluorescence. The analytes can be natural chromophores, which can emit fluorescent light once excited. Or the analytes may not emit fluorescence directly but can be labeled with fluorophores such as dyes. When resonators are used in the sensing elements 130, the signal from the fluorescence can be proportional to the Q-factor of the resonator(s) in the sensor. For sensors with ring resonators that have a high Q-factor, nonlinear excitation (e.g., two-photon or multi-photon excitation) can be used to excite biochemical samples whose absorption band is in the ultraviolet (UV) region of the electromagnetic spectrum.

In yet another example, the sensing elements 130 can be configured to perform absorption sensing. For linear waveguides, beam intensity can be directly monitored by the detector 140. For resonators, absorption measurements can be carried out by tuning the resonant wavelength across the absorption bands of the analytes for enhanced absorption path length. When the Q-factor of the ring resonator(s) is high (e.g., >10⁶, >10⁷, or >10⁸), absorption sensing can be carried out by monitoring the degradation of the resonant mode Q-factor. The degradation of the Q-factor can be caused by the extra loss induced by optical absorption.

In yet another example, the sensing elements 130 can be configured to perform Raman sensing. Raman spectroscopy in general relies on inelastic scattering, or Raman scattering, of monochromatic light photons with molecules. The light interacts with molecular vibrations, phonons, or other excitations in the system, resulting in an up-shift or down-shift of the light photon energy. The energy shift can be characteristic to certain molecules and therefore can be used to detect the presence of these molecules. Measurements by the ring resonator(s) in the sensor can also be combined with Raman spectroscopy to increase the Raman signals, which can lead to a gain of over two orders of magnitude. Using a high-Q ring resonator can also induce stimulated Raman emission, which can be employed for identification of species in liquid.

In yet another example, the sensing elements 130 can perform surface sensing, bulk sensing, or both. Surface sensing signals come from analytes in close proximity to the surface of the sensing elements 130 (much closer than the evanescent field decay length), whereas bulk sensing signals result from the optical change induced by the presence of the analytes in the whole region of the evanescent field.

The sensing elements 130 can include coatings on the surface to increase the sensitivity. In one example, the sensing elements 130 can include coatings of hygroscopic material to capture water vapor in the atmosphere. In another example, the sensing elements 130 include special enzymes to capture large molecules such as cells or proteins. In yet another example, the surface of the sensing elements 130 can textured or patterned to promote adhesion of the analyte molecules to the surface.

The two sensing elements 130a and 130b are normally sensitive to different wavelengths so as to detect different analytes or different transitions in the same analyte. Different sensitivity to different wavelengths can be achieved using various methods described below.

In one example, the two sensing elements 130a and 130b include different materials, each of which can be transparent to one spectral region. For example, one sensing element 130a can include silicon, which can have low loss for light at wavelengths over 2 μm, while the other sensing element 130b can include silicon oxide which can be transparent for light at wavelengths below 2 μm.

In another example, the two sensing elements 130a and 130b can have different dimensions (e.g., cross sectional dimensions) to propagate different optical modes of light beams. For example, one sensing element 130a can have a shape and size selected to allow single-mode propagation of one wavelength while the other sensing element 130b can have a shape and sizes selected allow single-mode propagation of another wavelength.

In yet another example, the two sensing elements 130a and 130b can have different coatings to capture different analytes. In yet another example, the two sensing elements 130a and 130b can have different sensing modes. For example, one sensing element 130a can be configured to carry out absorption sensing, while the other sensing element 130b can be configured to perform refractive index sensing. In yet another example, the two sensing elements 130a and 130b can have different types of sensors. For example, one sensing element 130a can include a linear waveguide while the other sensing element 130b includes a resonator sensor.

In yet another example, the two sensing elements 130a and 130b can include ring resonators. In this case, the two ring resonators can have different resonant wavelengths. Different resonant wavelengths can be achieved by, for example, using different materials, using different dimensions (e.g., diameter), or modulating the refractive indices of the ring resonators.

Various types of refractive index tuning methods can be used to obtain different resonant wavelengths for different ring resonators in the sensing elements 130. In one example, a piezo-electric element or other suitable element can be used to apply a mechanical force to the ring resonator to modulate the refractive index of the ring resonator. The mechanical force can be applied via, compression, bending, stretching, shearing, or any other means known in the art.

In another example, an electric field can be applied to the ring resonator to modulate the refractive index of the ring resonator. For instance, a modulator may apply the electric field via two electrodes, with one electrode attached to the top of the ring resonator and the other electrode attached to the bottom of the substrate. Alternatively or additionally, the electrodes can be attached to a perimeter of the ring resonator (e.g., one electrode on the inner diameter of the ring resonator and the other electrode on the outer diameter of the ring resonator, or any other configuration known in the art).

In yet another example, the temperature of the ring resonator can be varied to modulate the refractive index. For example, a modulator can include a semiconductor heater fabricated in thermal communication with (e.g., beside) the ring resonator. In another example, the modulator can include a semiconductor heater fabricated beneath the ring resonator in a substrate (e.g., within the substrate, or beneath the substrate). In yet another example, the modulator can include an external heater such as an oven that substantially encloses the entire apparatus 100 so as to uniformly change the temperature of the ring resonator.

In yet another example, an acoustic field can be applied to the ring resonator so as to modulate the refractive index (e.g., by sending a sound wave to compress the ring resonator). In yet another example, the modulator is configured to apply a magnetic field to the ring resonator so as to modulate the refractive index. In yet another example, the modulator is configured to apply an optical field to illuminate the ring resonator and change the refractive index.

In yet another example, the ring resonator includes chalcogenide glasses, in which case an optical field can be applied to modulate the refractive index. As understood in the art, chalcogenide glasses can exhibit several photo-induced effects, including photo-crystallization, photo-polymerization, photo-decomposition, photo-contraction, photo-vaporization, photo-dissolution of metals, and light-induced changes in local atomic configuration. These changes are generally accompanied by changes in the optical band gap and therefore optical constants. In addition, chalcogenide glasses also have strong third order nonlinear effects. Therefore, a modulator can adjust the optical properties of the ring resonator comprising chalcogenide glasses by applying a modulating optical field (separate from the light circulating in the ring resonator) on the ring resonator.

The detector 140 is optically coupled to the first sensing element 130a and the second element 130b. In one example, the first sensing element 130a and the second sensing element 130b can be combined via a directional coupler which is further coupled to the detector 140. In another example, the detector 140 can be coupled to the sensing elements 130 via an output switch (not shown in FIG. 1). The output switch can be substantially similar to the input switch 110, which can control which sensing element 130 illuminates the detectors 140 depending on the state of the phase change material in the output switch. In this case, potential interference of light beams from different channels can be further reduced.

The detector 140 can include one or more broadband sensing elements and/or spectrally selective narrowband sensing elements. For example, the detector 140 can include a spectrometer formed by a grating or other dispersive element that directs different spectral components of a broadband beam into different angles, each of which is monitored by a respective detector element in a detector array (e.g., a linear charged coupled device (CCD) or complementary metal-oxide-semiconductor (CMOS) sensing array) to yield the absorption spectrum of the absorbers evanescently coupled to the sensing elements 130. Alternatively or additionally, the detector 140 may include a single sensing element that detects the time-varying intensity of a spectrally swept (chirped) beam. Mapping the resulting time-varying intensity signal to the spectral sweep speed yields an absorption spectrum that can be used to identify any absorbers evanescently coupled to the sensor.

In one example, the detector 140 includes a direct detector (e.g., a single piece of CCD or CMOS detector) that senses the intensity of the incident beam. In another example, the detector 140 includes a differential detector, which can include two or more detector elements to derive useful information based on the difference between signals acquired from the detector elements. In yet another example, the detector 140 includes an interferometric detector, which can also include two or more detector elements but derive useful information based on the interference between the signals acquired from the detector elements.

The apparatus 100 can be used to detect various types of analytes 101 and 102. In one example, the analytes 101 and 102 can be gases. In another example, the analytes 101 and 102 can be liquids (e.g., acetone, gasoline, and alcohol, among others). In yet another example, the analytes 101 and 102 can be mixtures (e.g., atmosphere including various potential chemicals). In yet another example, the analytes 101 and 102 can be the same type of chemical, which includes at least two characteristic transitions and therefore at least two signature absorption/emission wavelengths. The two sensing elements 130a and 130b can be used to detect these two characteristic transitions so as to, for example, more reliably determine the presence or the absence of the analyte.

In one example, the apparatus 100 is used for quantitative sensing. In this case, the apparatus 100 can derive quantitative concentration of certain analyte nearby the sensing elements 130. In another example, the apparatus 100 is used for threshold sensing. In this case, the apparatus 100 returns positive detection signals (analyte found) if the concentration of certain analyte is at or above a threshold value and returns negative detection signals (analyte not found) if the concentration is below the threshold value.

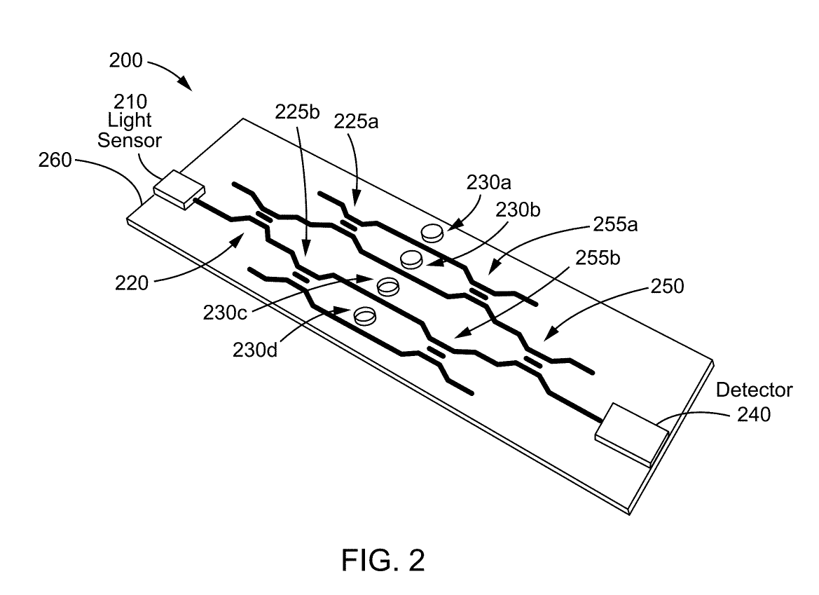

FIG. 2 shows a schematic of an apparatus 200 including a network of sensing elements 230a-230d coupled to a light source 210 and a detector 240 via multiple switches using phase change materials. The apparatus 200 includes a substrate 260 on which a light source 210 is disposed. An input switch 220 is disposed in optical communication with the light source 210 to receive light beams delivered by the light source 210. The input switch 210 has two output ports, each of which is coupled to a branch of sensors. The first output port is coupled to a first branch switch 225a and the second output port is coupled to a second branch switch 225b. Each branch switch 225a and 225b also has two output ports, each of which is coupled to a corresponding sensing element. More specifically, the first branch switch 225a is coupled to sensing elements 230a and 230b and the second branch switch 225b is coupled to sensing elements 230c and 230d. These four sensing elements 230a to 230d (collectively referred to as sensing elements 230) can have different operating wavelengths so as to detect different analytes or different transitions in the same analyte.

The outputs of the sensing elements 230a and 230b are coupled to a third branch switch 255a and the outputs of the sensing elements 230c and 230d are coupled to a fourth branch switch 255b. The two branch switches 255a and 255b are coupled to an output switch 250, which is further coupled to a detector 240 to detect the light beams transmitted through one or more of the sensing elements 230a to 230d.

In the apparatus 200, the input switch 220, the output switch 250, and the branch switches 225a, 225b, 255a, and 255b can be substantially similar to the input switch 120 shown in FIG. 1. In operation, light beams provided by the light source 210 can be transmitted to one of the sensing elements 230a to 230d by configuring the input switch 220 and the branch switches 225a and 225b. For example, if the first sensing element 230a is used, the input switch 220 can be adjusted to direct the light beam to the first branch switch 225a, which is adjusted to direct the light beam to the upper output port coupled to the sensing element 230a.

The output switch 250 and the two branch switches 255a and 255b can be used to control the light beam that enters the detector 240. For example, if the detector 240 is used to collect light from the fourth sensing element 230d, the branch switch 255b can be adjusted to direct light beams from the fourth sensing element 230d to the output switch 255b while blocking light beams from the third sensing element 230c from entering the output switch 255b. The output switch 250 then directs light beams transmitted through the branch switch 255b while blocks light beams transmitted through the branch switch 255a. The combination of output switch 250 and branch switches 255a/b can further reduce potential interferences between light beams from different sensing elements.

As shown in FIG. 2, the apparatus 200 is fabricated in a single substrate 260 (e.g., a semiconductor substrate). Therefore, the entire sensing system is monolithic and scalable. The multi-channel switches are realized by cascade of basic switching components. The system can share a light source 210 and guide the light to the target channel or sensing element. The apparatus 200 can be controlled electrically by logic components on the same chip or by signals from external controllers.

Switches in Directional Coupler Configurations

FIG. 3 shows a perspective view of a switch in directional coupler configuration that can be used in the systems 100 and 200. The switch 300 includes a phase change material 310 disposed between two substantially parallel waveguides 325a and 325b. The upper waveguide 325a is coupled to a first input waveguide 320a and a first output waveguide 330a. The lower waveguide 325b is coupled to a second input waveguide 320b and a second output waveguide 330b. The phase change material 310 is further coupled to two electrodes 340a and 340b, which can inject electric current through the phase change material 310 so as to change the state of the phase change material 310. The phase change material 310, the waveguides 320a/b, 325a/b, and 330a/b, and the electrodes 340a/b are disposed on a single substrate 360. The thickness of the phase change material 310 can be substantially similar to the thickness of the waveguides 325a and 325. For example, the thickness can be around about 200 nm to about 2 μm (e.g., 200 nm, 500 nm, 1 μm, 1.5 μm, or 2 μm).

In operation, light beams can be coupled to the switch 300 via one of the input waveguides 320a or 320b. The state of the phase change material 310 determines which output (330a or 330b) the light beam is transmitted to. For example, if the lower input waveguide 320b is used to receive the light beam, the light beam can be directed to the lower output waveguide 330b when the phase change material 310 is in the absorptive state. If output to the upper output waveguide 330a is desired, the electrodes 340a and 340b can inject an electric current to the phase change material 310 so as to change the phase change material 310 into the transmissive state. In this case, light beams (e.g., >95% or >98% of the beam energy) can be coupled from the lower input waveguide 320b into the upper output waveguide 330a.

The apparatus 300 shown in FIG. 3 uses a pair of electrodes 340a and 340b as an actuator to switch the phase change material 310 between one state and another. In practice, various other methods can be used to achieve the phase change.

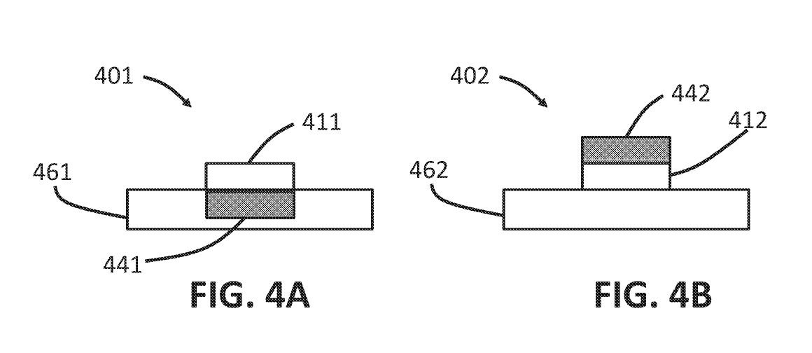

FIGS. 4A-4B show schematics of switches including actuators to change the phase of the phase change material. FIG. 4A shows the schematic of a switch 401 including a substrate 461 on which a phase change material 411 is disposed. An actuator 441 is disposed below the phase change material 411 to switch the state of the phase change material 411. The actuator 441 is within the substrate 461 as shown in FIG. 4A for illustrating purposes only. In practice, the actuator 441 can also be disposed below the substrate 461, i.e., the substrate 461 is sandwiched between the phase change material 411 and the actuator 461.

FIG. 4B shows the schematic of a switch 402 including a substrate 462 on which a phase change material 412 is disposed. An actuator 442 is disposed above the phase change material 412 to switch the states of the phase change material 412. The actuator 442 is in physical contact with the phase change material 412 for illustrative purposes only. In practice, the actuator 442 can be away from the phase change material 412. For example, the actuator 442 can in optical communication with the phase change material 412 via a transparent layer, or in thermal communication with the phase change material 412 via a thermal conductive layer.

Several types of actuators 441 and 442 can be used in the switches 401 and 402. In one example, the actuators 441 and 442 include a waveguide that can deliver a high power laser pulse to the phase change materials 411 and 412 to initiate the phase change. In another example, the actuators 441 and 442 include a pulsed laser head to deliver a laser pulse directly to the phase change material 411 and 412. In yet another example, the actuators 441 and 442 include heating elements (e.g., electrically driven hot plate or thermal-electrical cooler and/or heater) that can control local temperature to change the phase. The heating elements can include, for example, metal heaters, poly-silicon heaters, or any other heaters known in the art.

Directional Coupler Configurations Using Specially Engineered Silicon Nitrides

The waveguides 320a/b, 325a/b, and 330a/b in the switch 300 include a silicon nitride (SiNₓ) material specially engineered to reduce optical losses. The specially engineered SiNₓ material can be used to make the switch 300 shown in FIG. 3 by disposing a phase change material between two adjacent waveguides. This SiNₓ material can also be used to fabricate the sensing elements 130 and 230 shown in FIGS. 1-2.

SiNₓ films are known for their excellent performance in NIR micro-photonics owing to their extremely low optical loss, high optical nonlinearity, mechanical robustness and strong chemical stability. Moreover, SiNₓ can be compatible with very-large-scale integration (VLSI) processes and can be deposited on a variety of substrates. This makes SiNₓ a versatile potential candidate for a universal mid-IR platform.

However, one issue in fabricating SiNₓ devices for applications beyond λ=2.4 μm comes from the difficulty of depositing thicker crack-free films. In other words, to achieve low propagation loss in the mid-IR (λ>2.4 μm), mid-IR waveguides normally have a micron-scale thickness level so the wave is well confined inside the waveguide. However, typical stoichiometric silicon nitride Si₃N₄ generally has a high tensile stress greater than 1000 MPa. As a result, it can be challenging to fabricate Si₃N₄ films greater than 250 nm thick without cracking.

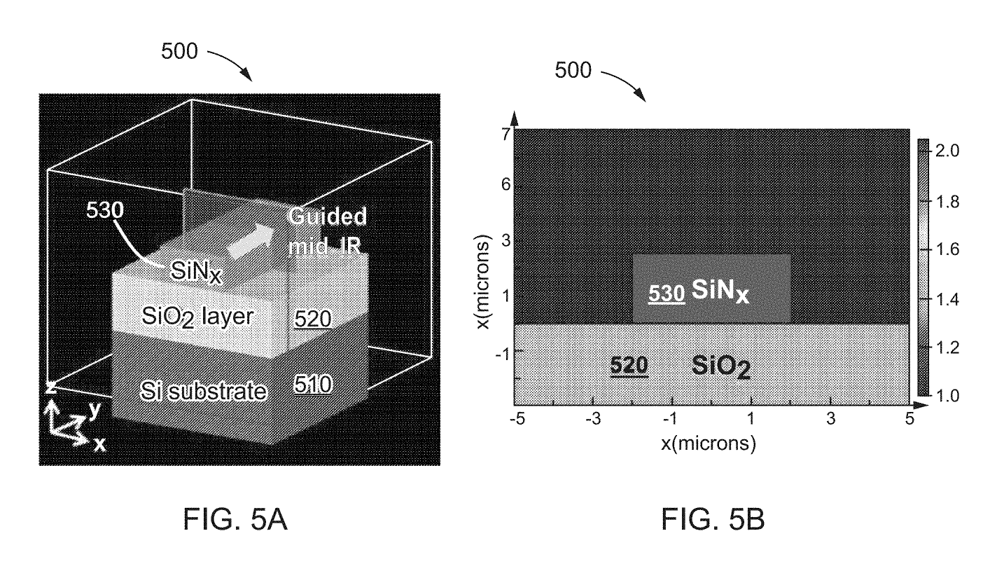

To reduce cracking, a different stoichiometric composition can be used to deposit the SiNₓ films. FIGS. 5A-5B show the perspective view and cross sectional view of a silicon nitride waveguide system using Si₃N₄ as the waveguide material. The system 500 includes a SiNₓ (Si₁₁N₉) waveguide 530 disposed on a SiO₂ layer 520, which is further disposed on a silicon substrate 510. The waveguide 530 has a height h=4 μm and a width w=2.5 μm. Underneath the SiNₓ waveguide 530 is a 4 μm thick buffer SiO₂ oxide 520 serving as an optical undercladding layer that prevents substrate leakage. FIG. 5B also shows the refractive index profile along the waveguide cross-section plane (x-z plane). Index of SiNₓ nsiNₓ is 2.05 obtained from ellipsometry measurements at λ=2.55 μm.

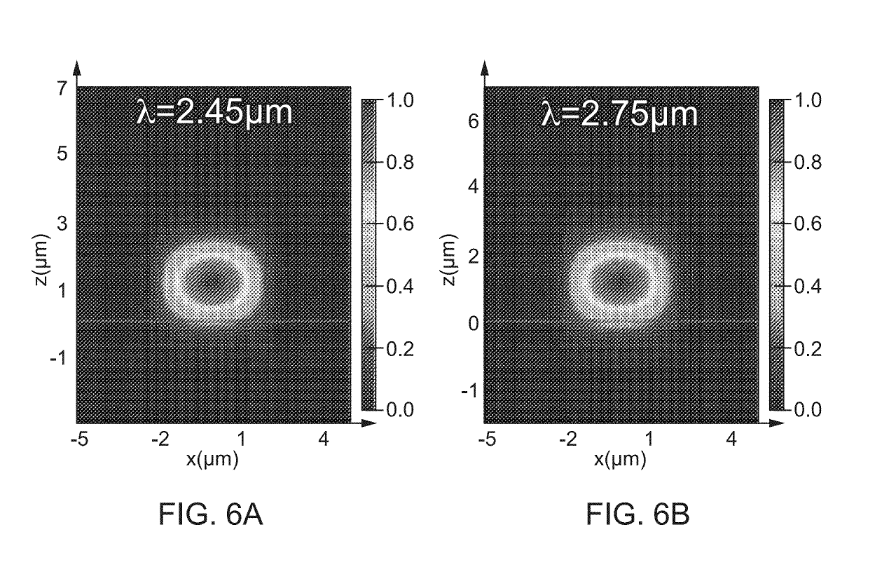

Two dimensional finite difference method (FDM) calculations can be employed to simulate the waveguide modes of the system 500. FIGS. 6A-6B show the calculated 2D mode images at λ=2.45 μm and λ=2.75 μm, respectively. In these two calculations, the calculated mode index is 1.96. A fundamental mode is obtained at both mid-IR wavelengths and the fundamental mode is well confined to the SiNₓ waveguide in both lateral (x) and vertical (y) directions. The intensity of the evanescent wave at the undercladding layer (z<0 μm) increases slightly as the wavelength increases from 2.45 μm to 2.75 μm.

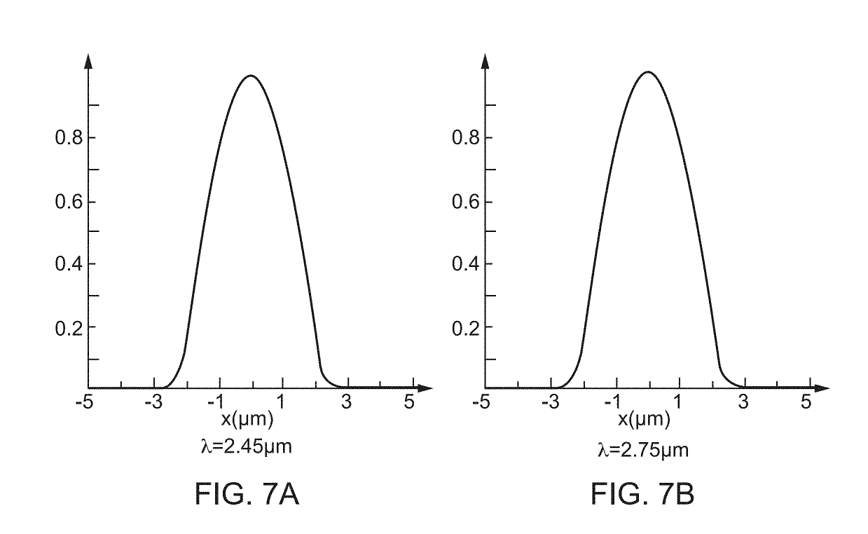

FIGS. 7A-7B show the calculated 1D intensity profile of the optical modes shown in FIGS. 6A-6B to better visualize the mode shapes. The calculations show that only one single Gaussian peak is found within the waveguide, indicating that the waveguide is single mode waveguide.

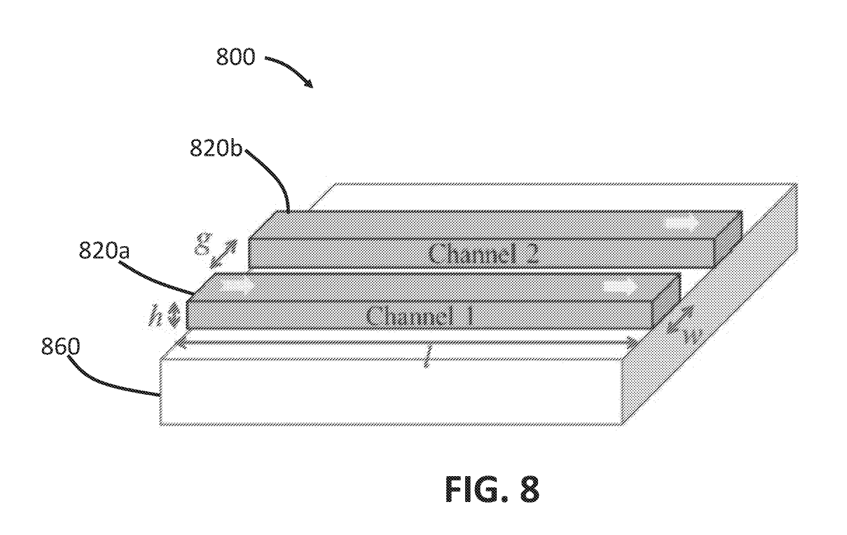

FIG. 8 shows a schematic view of a directional coupler including SiNₓ waveguides. The directional coupler 800 includes a first channel 820a and a second channel 820b, both of which are disposed on a substrate 860. The first channel 820a and the second channel 820b are made of the Si₁₁N₉ material. Each channel 820a and 820b has a length l, a width w, and a height h. The two channels 820a and 820b are separated by a gap g.

Performance of the directional coupler 800 can be simulated using finite-difference time-domain (FDTD) methods. In the simulation, the first channel 820a and the second channel 820b represent the two individual waveguides within one coupler. These two waveguides are identical. The width w, height h, and length l, are 4 μm, 2.5 μm, and 8 mm, respectively. One factor that influences the coupling efficiency is the separation g between channels. In this case, g is 700 nm for illustrative purposes.

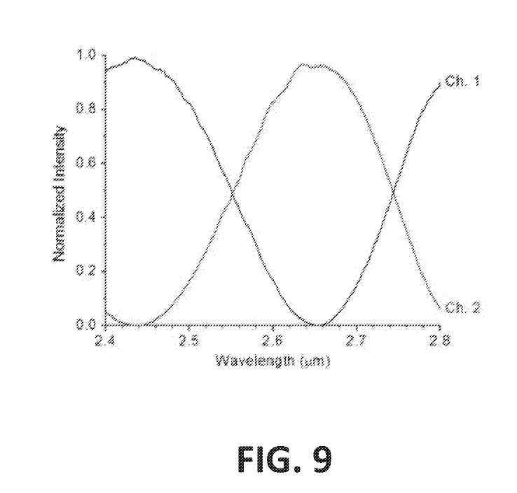

FIG. 9 is the intensity distribution between channel 1 and channel 2 calculated from λ=2.4 μm to λ=2.8 μm. The light output alternates as the wavelength shifts, and the intensity is sine/cosine relative to the probing wavelength.

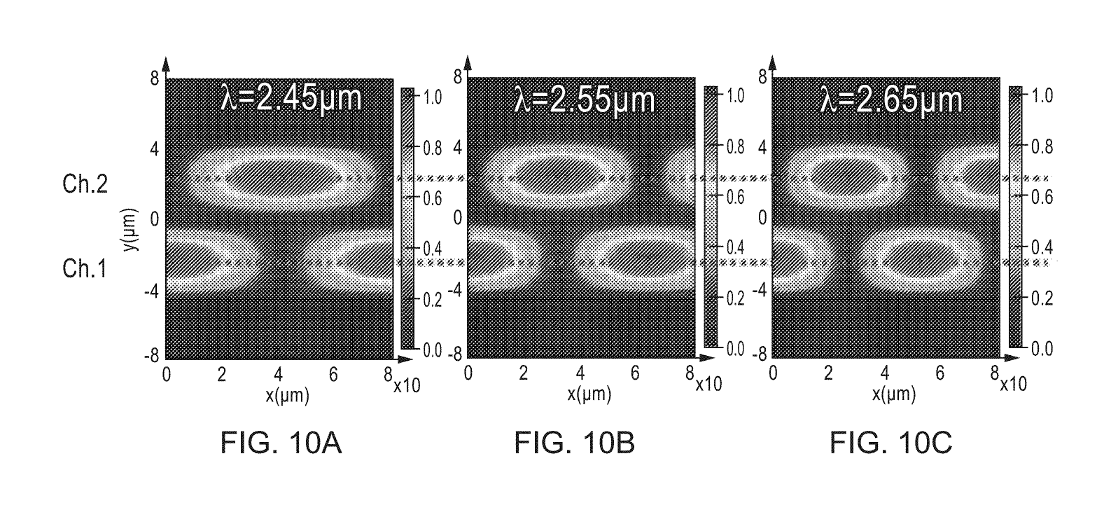

FIGS. 10A-10C show calculated spatial intensity profiles at different wavelengths in the directional coupler 800 to simulate 2D lightwave propagation and better understand how mid-IR light interacts with SiNₓ directional couplers. They show that the calculated guided mid-IR wave oscillates back and forth between the two waveguides before it finally reaches the output. As a result, the intensity distribution at the output facet (y=8 mm) can be determined by the input mid-IR wavelength. For instance, at λ=2.42 μm the maximum calculated intensity appears at channel 1, and then the intensities become equal at both channels when wavelength shifts to λ=2.55 μm. As the simulated wavelength is increased to λ=2.65 μm, the mid-IR light wave switches almost completely to channel 2. Furthermore, the guided mid-IR light retains its “fundamental mode” nature (single sharp spot), even after several rounds of coupling.

Methods of Fabricating SiNₓ Films

The buffer SiO₂ layer 520 show in FIG. 5 can be prepared by wet oxidation, in which the gases used are H₂/O₂ and a pyrogenic torch with a flow rate of 3000 sccm. The oxide film can be grown under atmospheric pressure and the temperature can be, for example, at 1100° C. Under these conditions, a deposition rate of 9 nm/min can be obtained.

The low stress SiNₓ films can be prepared by low-pressure chemical vapor deposition (LPCVD) techniques. Before loading the SiO₂ on the 4 inch Si substrates in the LPCVD furnace, the substrates can be cleaned using a standard Piranha solution, a mixture of sulfuric acid (H₂SO₄) and 30% hydrogen peroxide (H₂O₂) with volume ratio of 3:1, in order to remove any organic residues. For SiNₓ film growth, the silicon source can include dichlorosilane (DCS) and the nitrogen source can include ammonia NH₃. A high DCS:NH3 gas ratio of 3:1 can be used to produce Si-rich SiNₓ films. The deposition pressure can be around 200 mTorr and the reactor temperature can be about 825° C. After deposition, the first cool down time from 835° C. to 550° C. can be about 100 minutes and the second cool down time from 550° C. to room temperature can be about 20 minutes. A deposition rate of 10 nm/min can be obtained determined from film thickness characterization.

To fabricate the mid-IR planar devices on the Si-rich SiNₓ film, photolithography and inductively coupled plasma reactive ion etching (ICP-RIE) can be employed. Hexamethyldisilazane (HMDS) and a micron thick photoresist (e.g., Shipley 1813) can be initially coated on the SiNₓ film with a speed of, for example, 4000 revolutions per minute (rpm). The coated wafer can be baked. The baking temperate can be, for example, 115° C. and the baking time can be, for example, about 1 minute. Desired layouts including waveguides, splitters, and couplers can be defined by the photo-mask through UV patterning and then developed using MF-319 solution. These structures are then transferred into the SiNₓ layer through an optimized ICP-RIE etching process, which can last for about 15 minutes in Ar/H₂/CHF₃/CF₄ with a flow rates of 6/30/50/2 sccm respectively.

Experimental Results of SiNₓ Devices

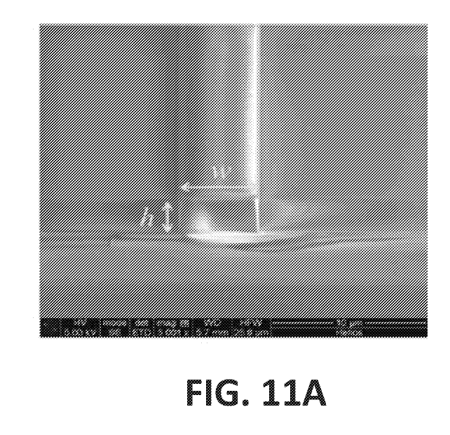

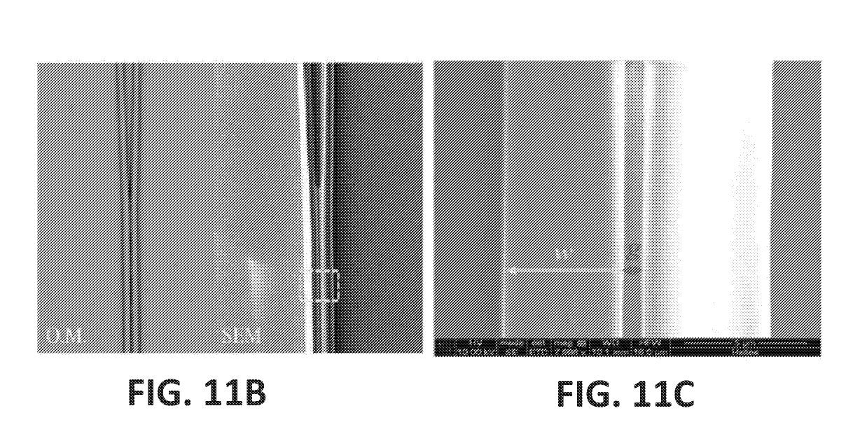

FIGS. 11A-11C are scanning electron microscopy (SEM) images of waveguides and directional couplers made from Si₁₁N₉. To characterize the height h, and width w, an SEM image of a waveguide is captured at a tilt angle of 54° as shown in FIG. 11A. A height h=4 μm and a width w=2.5 μm are obtained. In addition, the waveguide shows a smooth surface and sharp edges without bumps or indentations.

FIG. 11B shows SEM image of a directional coupler is. Two separate waveguides are merged into a coupling section with a coupling length of L. From the SEM image shown in FIG. 11C, the gap g between the adjacent waveguides is about 700 nm, along which no fusion is observed over the entire coupling length. The directional coupler also has a sharp gap edge and a constant gap width, thereby allowing well-resolved mid-IR light coupling.

More information can be found in Pao Tai Lin, et al., Low-Stress Silicon Nitride Platform for Mid-Infrared Broadband and Monolithically Integrated Microphotonics, Advanced Optical Materials, 2013, 1, 732-739, which is incorporated herein in its entirety.

Switches in Latch Configurations

As described above, switches that can be used in the systems 100 and 200 can have either a directional coupler configuration or a latch switch configuration. Switches in a directional coupler configuration are described above with reference to FIGS. 3-11. This section describes switches in a latch switch configuration with reference to FIGS. 12-15.

An ultra-subwavelength ON/OFF optical switch can be constructed based on variation of the attenuation of phase change materials. The switch can include a metal-dielectric-metal waveguide structure integrated with Ge₂Sb₂Te₅ (GST) chalcogenide glasses. There are two characteristics of GSTs that can help the construction of optical latches driven by electrical signals. First, GST materials can change their state from amorphous to crystalline by simple Joule heating. Second, accurate spatial location control can be obtained by lithographically patterned electrical contacts, thereby embedding the alignment into the switches. This latch switch can overcome two major challenges for electrically driven schemes: (i) metal contacts demonstrating low absorption, low resistance, and high temperature stability; and (ii) low loss optical wave guiding with very thin (tens of nanometers) GST films.

FIG. 12 shows a schematic of a latch switch including a phase change material. The latch switch 1200 includes a GST layer 1210 disposed between two gold electrodes 1240a and 1240b. The two electrodes 1240a and 1240b are electrically coupled to a power source 1250 that injects an electric current through the GST layer 1210 to change the phase of the GST layer 1210. A first SiO₂ layer 1220a is disposed on one side of the GST layer 1210 and a second SiO₂ layer 1220b is disposed on the other side of the GST layer 1210. The latch switch 1200 shown in FIG. 12 uses GST as the phase change material for illustrative purposes only. In practice, other phase change materials (e.g., any of the phase change materials described above) can be used.

Gold is used for the electrodes 1240a and 1240b for at least two reasons. First, gold can help the formation of an optical waveguide by Plasmon polaritonic resonance effect. Second, gold can also have good electrical contact.

Multi-physics software based on finite element method can be used to simulate the performance of the latch switch 1200. In the simulations, the operating wavelength is at 1550 nm. The refractive index of GST is set at 4.4+i0.098 for the amorphous state and 7.1+i0.78 for the crystalline state. The refractive index of the gold is 0.55+i11.51. The SiO₂ has a refractive index of 1.45. In addition, the thickness of the GST layer is 50 nm, which is typical of electronic phase change materials. The waveguide has a width w=30 nm (see FIG. 12). The thickness of the gold contacts t is 100 nm.

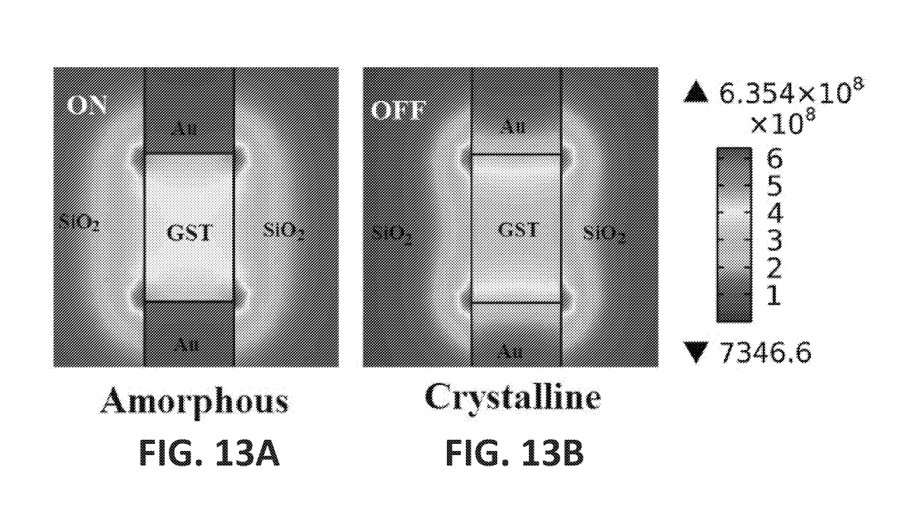

FIGS. 13A-13B show the calculated electric field of the optical mode in the latch switch 1200 shown in FIG. 12. FIG. 13A shows the electric field when the switch is ON, while FIG. 13B shows the electric field when the switch is OFF. The light is well confined in the waveguide due to the plasmonic resonance effect. The coupling between the GST and the metal can be enhanced along the interface for the OFF states since the refractive index of GST in the crystalline state is larger than in the amorphous state.

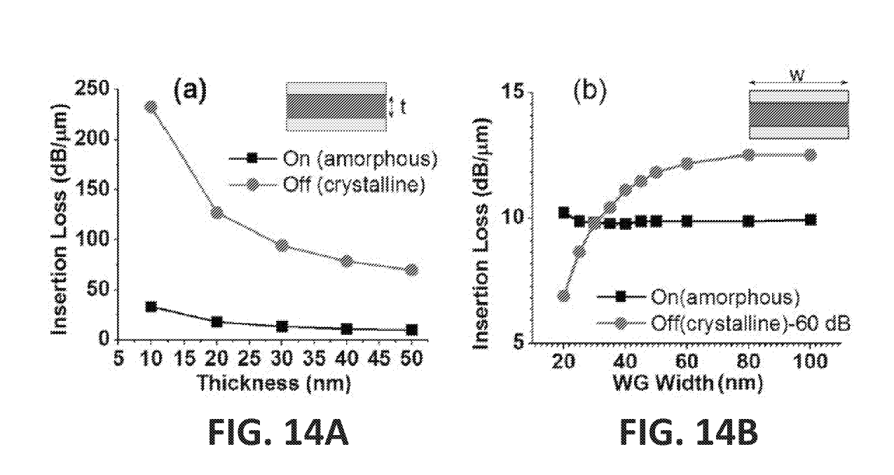

FIG. 14A shows the total insertion loss per unit of length of the latch switch 1200 as a function of the GST thickness from 10 to 50 nm (waveguide width is fixed to 30 nm). For both states, the insertion loss of the latch decreases with thicker GST films. However, the OFF state (crystalline GST) experiences a higher level of loss than the ON state (amorphous GST) as the GST film thickness decreases. This observation may be because SPP resonance between GST and the gold layer can be enhanced for the thinner film, therefore inducing a higher absorption loss.

FIG. 14B shows the variation of insertion loss per unit of length of the latch switch 1200 as a function of the waveguide core width from 20 to 100 nm (the thickness of the GST layer is fixed to 50 nm). In general, losses vary differently with waveguide width in the ON (amorphous GST) and OFF (crystalline GST) states. In the ON state, optical losses do not exhibit significant changes with waveguide width w. However, in the OFF state, higher width dependence is seen as higher losses are exhibited because coupling with the lossy metal layer may be stronger.

FIG. 14B also shows that the insertion loss for the ON state changes slowly with the variation of waveguide width w, while the insertion loss for the OFF state is significantly increased with the waveguide width w. This indicates that a larger waveguide width w can result in a higher extinction ratio between ON and OFF states. On the other hand, a wider waveguide consumes more energy to drive the phase change of the material. Therefore, the waveguide width w may be selected based on the desired energy consumption and extinction ratio.

A Joule heat model that couples both electrical and thermal properties of the materials (GST and gold) can be employed to find the energy cost and operation time of the latch switch 1200. In the simulation, GST in crystalline state has conductivity of 2.77×10³ Ω⁻¹m⁻¹ and heat capacity of 210 JKg⁻¹K⁻¹, GST in amorphous state has conductivity of 0.1 Ω⁻¹m⁻¹ and heat capacity of 210 JKg⁻¹K⁻¹, and gold has conductivity of 4.52×10⁷ Ω⁻¹m⁻¹ and heat capacity of 129 JKg⁻¹K⁻¹. The thermal conductivity of GST in crystalline state, GST in amorphous state, and gold is 0.24, 0.28, and 318 Wm⁻¹K⁻¹. In addition, GST thickness of 50 nm is used.

The analysis can begin with the switching-off process, i.e., the transition of the GST material from the amorphous to crystalline state. Without being bound by any particular theory or mode of operation, the process can be considered as a recrystallization under high temperature, yet below the melting point of the material. The resistivity of amorphous GST can be so high (typically about 10 Ω·m) that there is almost no measurable current below the threshold voltage. However, amorphous GST has a threshold voltage, normally occurring at a certain current density, above which it becomes conductive without any phase change and consequently can generate Joule heating that induces the phase change. The typical switching-off time (or “Set” process in a phase-change material) can be, for example, about 100 ns. This time can be much longer than the typical switching-on time (or “Reset” process), which is around 1 ns. This indicates that the energy consumption of the switching-off process can be dominant, therefore requiring only an optimization of the energy cost for the switching-off process to optimize the energy consumption of the entire switch.

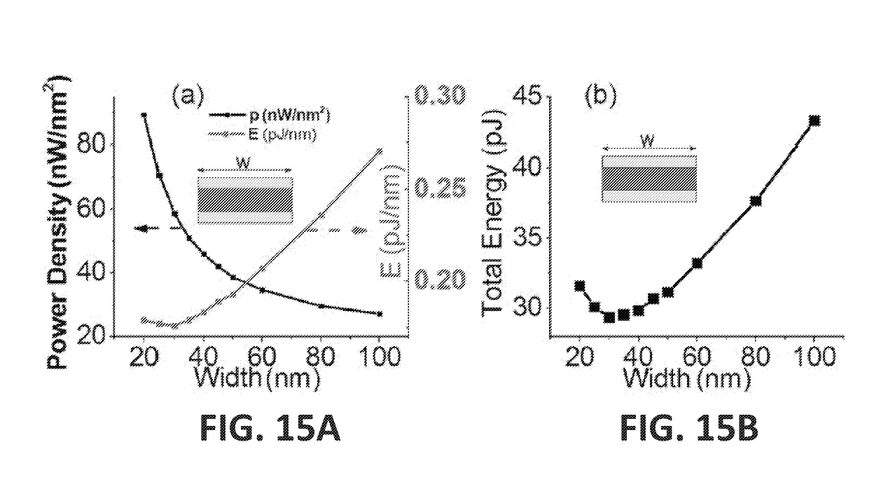

FIGS. 15A-15B show calculated results of the power density and total energy consumption of the latch switch 1200. As shown in FIG. 15A, the electrical power density for the heating process decreases with increasing waveguide width w because the effective surface increases, thus making the heat transfer into the substrate faster. However, the energy cost per unit of length of the latch switch increases with increasing waveguide width w because of the larger volume of GST that needs to be crystallized. On the other hand, as shown previously, a larger width w can also result in larger extinction ratio per unit of length of the device.

FIG. 15B shows the total energy consumed for the switching-off process given a desired absorption change of 10 dB between the ON and OFF states. In FIG. 15B, the optimal width w for a 50 nm thick GST is 30 nm. With a length of 167 nm, the latch switch can have an insertion loss of 1.64 when it is on and 11.64 dB when it is off, with operation energy of only 30 pJ. There can be several ways to increase the extinction ratio between ON/OFF states, such as using longer device length, thinner GST layers, or larger waveguide width. For example, the extinction ratio can increase to 20 dB if the device length is extended from 167 nm to 334 nm.

For the switching-on process, the crystalline GST can melt into liquid and then can be rapidly quenched, typically within a few nanoseconds, which results in an amorphization of the structure. Assuming that the conductivity of the crystalline GST does not change before melting and applying a voltage of 3.5 V, a current density of 1.94×10⁴ kA/cm² can be calculated. After 57 ps, the maximum temperature reaches the melting temperature (883 K). With further heating, GST can melt and the latent heat (1.37×10⁵ J/kg) can consume most of the input power, pinning the temperature to the melting point. By treating the conductivity of GST as constant and assuming that all the heating energy goes into latent heat after 57 ps, the total phase transition time can be only 0.12 ns. The energy consumption for the phase transition is 0.4 pJ, which is much smaller than that of the switching-off process. So the energy consumption per cycle (including both the switching-on and switching-off processes) is about 30.4 pJ.

A figure-of-merit (FOM) can be defined as power times the footprint of the device to further characterize the latch switch 1200. The FOM of the latch switch 1200 can scale down with operation frequency, due to the intrinsic self-holding nature of the switch. The operation time for the switch is about 100 ns. The overall energy cost of the device (including packaging) is estimated to be similar to phase change material devices, which is 290 pJ per cycle. By comparing the latch switch 1200 with other switches based on different schemes such as optical Microelectromechanical systems (MEMS), silica-based Mach-Zehnder interferometer (MZI), thermooptical, electroholographic, Semiconductor Optical Amplifier (SOA)-based, acousto-optic, and microdisks, it can be seen that the FOM of the latch switch 1200 within the frequency cut-off can be higher than most of other optical switches.

More information can be found in Jianwei Mu, et al., Towards Ultra-Subwavelength Optical Latches, Applied Physics Letters, 103, 043115 (2013), which is incorporated herein in its entirety.

Conclusion

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of designing and making the technology disclosed herein may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes (outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. (canceled)

2. An apparatus for sensing a first analyte and a second analyte, the apparatus comprising:
a light source to transmit a light beam;
an input switch, in optical communication with the light source, to receive the light beam from the light source, the input switch comprising a phase change material having a first state and a second state;
a first sensing element, in optical communication with the input switch, to receive the light beam from the input switch when the phase change material is in the first state and to produce a first change in the light beam in response to a presence of the first analyte;
a second sensing element, in optical communication with the input switch, to receive the light beam from the input switch when the phase change material is in the second state and to produce a second change in the light beam in response to a presence of the second analyte;
an output switch, in optical communication with the first sensing element and the second sensing element, comprising additional phase change material switchable between the first state and the second state; and
a single-element detector, in optical communication with the output switch, to detect the first change in the light beam when the additional phase change material is in the first state and to detect the second change in the light beam when the additional phase change material is in the second state.

3. The apparatus of claim 2, wherein the light source is configured to emit the light beam with at least one spectral component at a wavelength in a range of about 2 μm to about 7 μm.

4. The apparatus of claim 2, wherein the phase change material comprises Ge₂Sb₂Te₅.

5. The apparatus of claim 2, wherein the input switch comprises:
a first electrode disposed on a first side of the phase change material; and
a second electrode disposed on a second side of the phase change material and configured to inject an electric current through the phase change material to the first electrode so as to switch the phase change material between the first state and the second state.

6. The apparatus of claim 2, wherein the input switch comprises a heater, in thermal communication with the phase change material, to heat the phase change material so as to switch the phase change material between the first state and the second state.

7. The apparatus of claim 2, wherein the input switch comprises:
a first semiconductor waveguide disposed on a first side of the phase change material in optical communication with the first sensing element; and
a second semiconductor waveguide disposed on a second side of the phase change material in optical communication with the second sensing element.

8. The apparatus of claim 2, wherein the first sensing element comprises a first resonator having a first resonant frequency and the second sensing element comprises a second resonator having a second resonant frequency different from the first resonant frequency.

9. The apparatus of claim 8, wherein the first resonator is a first ring resonator having a quality factor greater than about 10⁶.

10. The apparatus of claim 8, wherein the first resonator is a first ring resonator having a tunable refractive index.

11. The apparatus of claim 8, wherein the first resonator comprises a section of linear waveguide between a pair of reflectors.

12. The apparatus of claim 2, wherein the input switch is in a network of input switches and the output switch is in a network of output switches, the first sensing element and the third sensing element are in an array of sensing elements, and the single-element detector is configured to be coupled to each sensing element in the array of sensing elements depending on a state of the network of output switches.

13. The apparatus of claim 2, wherein the input switch is a latching switch.

14. The apparatus of claim 2, wherein the light beam is a chirped beam, wherein the single-element detector element detects a time-varying intensity of the light beam in response to the presence of the first analyte or the second analyte to identify an absorption spectrum associated with the first analyte or the second analyte.

15. The apparatus of claim 2, wherein the single-element detector is a spectrally selective single-element detector.

16. A method of sensing a first analyte and a second analyte, the method comprising:
transmitting a light beam through an input switch comprising first phase change material in a first state to a first sensing element, the light beam undergoing a first change in response to a presence of the first analyte on the first sensing element;
coupling the light beam from the first sensing element to a single-element detector via an output switch comprising second phase change material in the first state;
detecting the first change in the light beam with the single-element detector;
changing the first phase change material from the first state to a second state;
changing the second phase change material from the first state to a second state;
transmitting the light beam through the input switch to a second sensing element, the light beam undergoing a second change in response to a presence of the second analyte on the second sensing element;
coupling the light beam from the second sensing element to the single-element detector via the output switch; and
detecting the second change in the light beam with the single-element detector.

17. The method of 16, further comprising:
emitting the light beam with at least one spectral component at a wavelength in a range of about 2 μm to about 7 μm.

18. An apparatus for sensing a first analyte and a second analyte, the apparatus comprising:
a light source to transmit a light beam;
a latching input switch, in optical communication with the light source, to receive the light beam from the light source, the latching input switch comprising a phase change material having a first state and a second state, the phase change material disposed between a pair of metal electrodes configured to inject a current into the phase change material, the current switching the phase change material between the first state and the second state;
a first sensing element, in optical communication with the latching input switch, to receive the light beam from the latching input switch when the phase change material is in the first state and to produce a first change in the light beam in response to a presence of the first analyte;
a second sensing element, in optical communication with the latching input switch, to receive the light beam from the latching input switch when the phase change material is in the second state and to produce a second change in the light beam in response to a presence of the second analyte; and
a single-element detector, in optical communication with the first sensing element and the second sensing element, to detect at least one of the first change in the light beam or the second change in the light beam.

19. The apparatus of claim 18, wherein the light beam is a spectrally swept beam and the single-element detector detects a time-varying intensity of the light beam in response to the presence of the first analyte or the second analyte to identify an absorption spectrum associated with the first analyte or the second analyte.

20. The apparatus of claim 18, wherein the single-element detector is a spectrally selective element.
